(19) United States
(12) Patent Application Publication
(10) Pub. No.: US 2007/0154540 A1
(43) Pub. Date: Jul. 5, 2007

(54) Composition for treatment of osteoarthritis containing apigenin as chondroregenerative agent

(75) Inventors: Chang Shin Park, Incheon (KR); Ju Hee Kang, Incheon (KR); Gyoung Mi Kim, Seoul (KR)

Correspondence Address: FISH & RICHARDSON PC, P.O. BOX 1022, MINNEAPOLIS, MN, 55440-1022, US

(73) Assignee: KMSI CO., LTD., Yeonso-gu (KR)

(21) Appl. No.: 10/575,796

(22) PCT Filed: Oct. 15, 2004
(86) PCT No.: PCT/KR04/02653

§ 371 Date: Nov. 28, 2006

(30) Foreign Application Priority Data
Oct. 15, 2003 (KR) 10-2003-00717777

(51) Int. Cl. A61K 9/48; A61K 9/20; A61K 31/353

(52) U.S. Cl. 424/451; 514/456; 424/464

(57) ABSTRACT

Disclosed is a novel use of apigenin as a chondroregenerative agent, which has the effects of reducing elevated of cartilage destruction markers including total synovial fluid volume and proteoglycan, total proteins and prostaglandin in a synovial fluid, improving the condition of synovial cells, and regenerating cartilage. Also, the present invention discloses a therapeutic agent for osteoarthritis comprising apigenin as an agent regenerating articular cartilage, and a method of treating osteoarthritis using such a therapeutic agent.

### TECHNICAL FIELD

The present invention relates to a novel use of apigenin as a chondroregenerative agent, which has the effects of reducing elevated levels of cartilage destruction markers including total synovial fluid volume and proteoglycan, total proteins and prostaglandin in a synovial fluid, improving the condition of synovial cells, and regenerating cartilage. Also, the present invention is concerned with a therapeutic agent for osteoarthritis comprising apigenin as an agent regenerating articular cartilage, and a method of treating osteoarthritis using such a therapeutic agent.

### BACKGROUND ART

Arthritis is a common disease that can affect anybody at any age, and its incidence increases with age. In Korea, about 20% of the overall population suffers from arthritis. There are more than one hundred different types of arthritis, and osteoarthritis is the most common type of all arthritic conditions.

Osteoarthritis involves the deterioration of cartilage in the joints. Over time, the cartilage, covering the ends of bones in a joint, begins to break down and may wear away entirely, and the bones will rub together, causing pain. Due to pain in a joint, the surrounding muscle is used less, and muscle strength is thus weakened. Osteoarthritis, known as degenerative arthritis in the past, is a commonly occurring joint disease that occurs after abnormality or damage of joints or without joint damage.

The prevalence of osteoarthritis is similar in men and women. However, in women, a greater number of joints are affected, while men suffer from a higher frequency of hip joint invasion. The risk factors of osteoarthritis include aging, obesity, congenital dysplasia of the hip, trauma, a history of arthritis, particular job groups and heredity. Osteoarthritis itself does not greatly affect one's life, but chronic osteroarthritis sustaining for a long period of time causes pain and deformity of the joints and thus reduces the quality of life. In particular, osteoarthritis in the knees is known as a major cause of chronic disability. Recently, many studies have successfully developed various drugs and treatment methods, which are capable of reducing pain and joint deformity due to osteoarthritis.

In osteoarthritis, cartilage in the joints is damaged and wears away. This allows bones under the cartilage to rub together. As time goes by, the bones are damaged, and also may become deformed, thus causing pain and other several symptoms. Two factors are likely to be important in the etiology and pathogenesis of osteoarthritis: one is excess weight that puts extra stress on joints and thus damages tissues in the joints, the condition of cartilage or bones of the joints being normal; and the other is weakness of cartilage or bones in the joints under normal loads. Also, osteoarthritis occurs in about 50% of people over the age of 60 and about 70% of the elderly over the age of 65. Thus, the most important risk factor is likely to be aging.

Osteoarthritis occurs in joints mainly in the knees, hips, spine and fingers. The major symptoms include pain and joint deformity, and affected joints display edema, hot flashes and abnormal enlargement of joints.

Recently, various drugs and treatment methods have been developed and used for the treatment of osteoarthritis. The main goals of the treatment are to relieve pain, maintain the functions of the joints and prevent disability due to the functional disorder of the joints.

For osteoarthritis exhibiting only pain at early stages, the treatment methods are focused on relieving the pain by administering a simple analgesic. When osteoarthritis progresses with prolonged pain, anti-inflammatory agents having strong anti-inflammatory effects are used. However, upon long-term administration, these anti-inflammatory agents adversely affect the stomach, liver and kidney and also suppress the ability of chondrocytes to regenerate. Due to these side effects, the anti-inflammatory agents should be used with caution.

In detail, osteoarthritis has been treated using anti-inflammatory substances of the corticosteroid type, for example, hydrocortisone and betamethasone, which function to inhibit prostaglandin synthesis. However, upon oral administration, these steroid hormones may have temporary therapeutic efficacy, but, due to their serious side effects, they should be used with special caution and basically administered not by oral administration but by intra-articular injection.

The most commonly used method for treating arthritis is to administer a drug. To date, a large number of drugs having analgesic and anti-inflammatory effects have been developed, which include nonsteroidal anti-inflammatory drugs (NSAIDs) such as diclofenac, aspirin and ibuprofen.

However, NSAIDs commonly used for the treatment of arthritis have side effects of damaging the gastrointestinal tract, especially the stomach, by inhibiting both cyclooxygenase 1 (COX-1), which functions to protect the stomach wall, and cyclooxygenase 2 (COX-2), which causes pain and inflammation. In addition, when administered for a long period of time, NSAIDs cause soreness or ulcers in the stomach or intestine, eventually resulting in the formation of holes. The developed ulcers may block the exit of the stomach, thus causing vomiting and weight loss, as well as bleeding in the stomach and duodenum. In addition to NSAIDs, different-class therapeutic agents against arthritis, such as COX-2 inhibitors not inhibiting COX-1, have been developed, which have excellent analgesic effects and mild side effects with respect to causing the formation of ulcers and bleeding.

On the other hand, the treatment of osteoarthritis that is the most common phenomenon caused by aging was, in the past, focused only on relieving pain and inflammation without fundamental treatment. Recently, studies associated with the delay or inhibition of the progress of arthritis based on reducing the destruction of chondrocytes have good results.

For example, U.S. Pat. No. 6,610,750 discloses a method for treating osteoarthritis by delaying the progression of the destruction of joint cartilage using rhein and its ester derivative, diacerein.

U.S. Pat. No. 5,591,740 discloses a method of treating osteoarthritis by slowing the joint deterioration and cartilage degradation using debromohymenialdisine isolated from the marine sponge Hymeniacidon.

In addition, based on the finding that the protein tyrosine kinase inhibitors, such as genistein, herbimycin A, 4,5-dianilinophthalimide, tyrphostin AG82 and tyrphostin AG556, slow interleukin-1-induced cartilage degradation, U.S. Pat. No. 6,552,066 discloses a method of treating osteoarthritis using the protein tyrosine kinase inhibitors.

Further, U.S. Pat. No. 5,650,433, which corresponds to Japanese Pat. Publication No. 07-025761, discloses a chondroprotective agent containing a flavonoid compound, a glycoside thereof or a stereoisomer thereof, which has an inhibitory effect against the depletion of proteoglycan that is a major component of the cartilage matrix, and a method of treating arthropathy by reducing the cartilage destruction through administration of the chondroprotective agent.

However, the methods disclosed in the aforementioned patents and other methods of treating osteoarthritis using drugs known up to date delay or inhibit the progress of the cartilage destruction, but do not include therapeutic agents having a chondroregenerative effect and thus being capable of restoring damaged cartilage.

Recently, to avoid the difficulty associated with the protection of the cartilage with osteoarthritis therapy using drugs having side effects, methods not employing drugs have been tried. For example, the regeneration of the cartilage tissue has been attempted using mesenchymal stem cells (EP Laid-open Publication No. 0989855A1 and U.S. Pat. Application Laid-open Publication No. 20020110544A1).

To date, in osteoarthritis therapy, only treatment methods of providing anti-inflammatory and analgesic effects or delaying or inhibiting cartilage destruction are used to slow the clinical progress of osteoarthritis. Thus, there is a need for the development of a novel method of treating osteoarthritis caused by cartilage degradation, which is capable of regenerating cartilage tissue or improving the condition of cartilage tissue.

This need is driven by the fact that cartilage tissue consists of about 95% water and extracellular cartilage matrix and only 5% chondrocytes, and that chondrocytes have the longest cell cycle in the body and are thus very difficult to proliferate (osteoarthritis and neurogenic arthropathy, pages 487 and 492, the Merck Manual, 17th English Edition/First Korean edition, 2003). Thus, a significant advance in the medical field may be achieved by finding a substance capable of promoting the regeneration of cartilage with no side effects by in vivo tests, using a proliferation inducer to proliferate chondrocytes isolated from an osteoarthritis patient and implanting the proliferated chondrocytes into the patient.

The present inventors found that a naturally occurring flavonoid compound, apigenin, has a novel use as an agent promoting the proliferation of chondrocytes and thus regenerating cartilage tissue, as well as the known effect of delaying the destruction of chondrocytes, thus leading to the present invention.

### DISCLOSURE OF THE INVENTION

In a first aspect, the present invention provides a composition for regenerating cartilage, comprising apigenin, in an amount effective for promoting the proliferation of chondrocytes with no cytotoxicity, and a pharmaceutically acceptable carrier.

In the first aspect, the composition for regenerating cartilage according to the present invention comprises apigenin in an amount resulting in a concentration of 0.1 μM to 100 μM in an articular cartilage and a synovial fluid of a patient. In this dosage range, apigenin has a biochemical effect of reducing indicators or markers of osteoarthritis: increased joint synovial fluid volume and increased levels of proteoglycan, total proteins and prostaglandin in a synovial fluid, and has an additional effect of improving articular cartilage by improving the condition of synovial cells.

Preferably, the effective amount of apigenin contained in the composition for regenerating cartilage according to the present invention is administered in an amount resulting in a concentration of 1 μM to 80 μM in an articular cartilage and a synovial fluid of a patient.

In a second aspect, the present invention provide a therapeutic agent for osteoarthritis for regenerating cartilage, comprising the composition for regenerating cartilage according to the first aspect and a pharmaceutically acceptable excipient.

In the second aspect, the therapeutic agent for osteoarthritis is in the form of orally administrable preparations such as solutions, capsules, granules, tablets or pills; topically applicable preparations such as ointments or transdermally administrable preparations; or injectable preparations.

When the therapeutic agent for osteoarthritis according to the present invention is in the form of oral preparations, apigenin is administered in a daily dosage of 10 mg to 1000 mg.

When the therapeutic agent for osteoarthritis according to the present invention is in an ointment formulation, apigenin is administered in a daily dosage of 1 mg to 100 mg.

When the therapeutic agent for osteoarthritis according to the present invention is in an injectable formulation, apigenin is administered in a daily dosage of 0.1 mg to 10 mg.

In a third aspect, the present invention provides a method of treating osteoarthritis by regenerating articular cartilage, comprising administering a therapeutic agent for osteoarthritis, comprising the composition for regenerating cartilage according to the first aspect and a pharmaceutically acceptable excipient, to an osteoarthritis patient.

### BRIEF DESCRIPTION OF THE DRAWINGS

The above and other objects, features and other advantages of the present invention will be more clearly understood from the following detailed description taken in conjunction with the accompanying drawings, in which:

FIG. 1 photographically shows the cartilage of osteoarthritis-induced rabbits, wherein a control group (A) has been treated with physiological saline, and a test group (B) has been treated with apigenin according to the present invention;

FIG. 2 is a graph showing changes in total synovial fluid volume according to apigenin administration, according to the present invention, in osteoarthritis-induced rabbits (normal: total synovial fluid volume in normal rabbits; OA: total synovial fluid volume immediately after osteoarthritis induction; +: results in right legs treated with apigenin or physiological saline; −: result in non-treated left legs);

FIG. 3 is a graph showing changes in total proteoglycan levels in joint synovial fluids according to apigenin administration, according to the present invention, in osteoarthritis-induced rabbits (normal: total proteoglycan levels in joint synovial fluids in normal rabbits; OA: total proteoglycan levels in joint synovial fluids immediately after osteoarthritis induction; + and −: the same meaning as in FIG. 2);

FIG. 4 is a graph showing changes in total protein levels in joint synovial fluids according to apigenin administration, according to the present invention, in osteoarthritis-induced rabbits (normal: total protein levels in joint synovial fluids in normal rabbits; OA: total protein levels in joint synovial fluids immediately after osteoarthritis induction; + and −: the same meaning as in FIG. 2);

FIG. 5 is a graph showing changes in total prostaglandin E2 (PGE2) levels in joint synovial fluids according to apigenin administration in osteoarthritis-induced rabbits (normal: total PGE2 levels in joint synovial fluids in normal rabbits; OA: total PGE2 levels in joint synovial fluids immediately after osteoarthritis induction; + and −: the same meaning as in FIG. 2);

FIG. 6 is a graph showing changes in collagen levels in joint synovial fluids according to apigenin administration in osteoarthritis-induced rabbits (normal: total collagen levels in joint synovial fluids in normal rabbits; OA: total collagen levels in joint synovial fluids immediately after osteoarthritis induction; + and −: the same meaning as in FIG. 2);

FIG. 7 is a graph showing changes in Mankin scores according to apigenin administration in osteoarthritis-induced rabbits (+ and −: the same meaning as in FIG. 2);

FIGS. 8A and 8B photograpically show the results of staining of cartilage in joints of osteoarthritis-induced rabbits administered with apigenin (FIG. 8A) and physiological saline (FIG. 8B) (H&E: treatment groups stained with hematoxylin and eosin; Saf-O: treatment groups stained with safranin-O)

FIG. 9 photograpically shows the results of staining of the synovial memebrane in joints of osteoarthritis-induced rabbits administered with apigenin (A) and physiological saline (B);

FIG. 10 is a graph showing the changes in the number of synovial membrane lining cells in osteoarthritis-induced rabbits according to apigenin administration;

FIG. 11 shows the results of Western blotting for changes in expression levels of iNOS, COX-2 and IκBα in a macrophage cell line derived from mice, RAW 264.7, during LPS-induced inflammation according to apigenin administration, according to the present invention, wherein β-actin is used as an internal marker;

FIG. 12 is a photograph showing the results of EMSA (Electrophoretic Mobility Shift Assay) for detecting the binding between a transcription factor, NFκB, and a specific gene in a macrophage cell line derived from mice, RAW 264.7, according to apigenin administration according to the present invention; and

FIG. 13 schematically illustrates the process of inducing osteoarthritis and collecting synovial fluids according to the time.

### BEST MODE FOR CARRYING OUT THE INVENTION

The present invention provides a novel use of apigenin for treating osteroarthritis based on its effects of delaying the destruction of chondrocytes and regenerating cartilage by stimulating the proliferation of chondrocytes.

Apigenin has been known to have anticancer effects such as cytotoxicity and cell proliferation-inhibiting activity. In contrast to these known effects of apigenin, the present inventors found that apigenin has an effect of regenerating cartilage by stimulating the proliferation of chondrocytes with no cytotoxicity in an effective dosage within a specific concentration range. That is, the chondroregenerative effect of apigenin was found to have a direct relation to an accurate dosage and a final amount applied to a target site. In detail, when used in an amount resulting in a concentration of 0.1 to 100 μM, and preferably 1 to 80 μM, in an articular cartilage and a synovial fluid, apigenin was found to have overall effects on articular cartilage regeneration, which include effects of inhibiting the activities of inflammation-associated enzymes and inhibiting the production of pain inducers in a cellular test, biochemical effects of reducing joint synovial fluids and levels of proteoglycan, total proteins and prostaglandin in synovial fluids in a test using an arthritis-induced animal model, and effects of regenerating cartilage and improving the condition of synovial cells upon a histological examination of the arthritis-induced animal model. These results indicate that apigenin has excellent activity as an agent regenerating articular cartilage. Based on this finding, the present invention provides a novel use of apigenin as an agent that prevents or inhibits osteoarthritis, as well as an agent that improves or treats osteoarthritis.

Hereinafter, the present invention will be described in more detail.

Osteoarthritis is a degenerative joint disease that is the oldest and most common type of arthritis, and is characterized by the destruction of articular cartilage. Osteoarthritis is an inevitable consequence of aging, and obesity may cause osteoarthritis in the knee joints. The risk of developing osteoarthritis may increase if the joints are injured from repeated use through activities such as sports or work or from accidents. Also, in knee osteoarthritis, when osteoarthritis occurs in one knee, a patient relies more on the other knee to avoid pain, thus increasing the possibility of additionally developing osteoarthritis in the other knee. Thus, in a patient with osteoarthritis in both knees, the improvement of osteoarthritis in one knee can reduce the weight applied to the other knee, and thus, the risk of worsening the condition of osteoarthritis in the other knee is reduced, resulting in a positive effect on alleviation of the previously developed osteoarthritis.

Since osteoarthritis has a different etiology from rheumatoid arthritis that is an autoimmune disease caused by inflammation, a substance effective in rheumatoid arthritis is not effective in osteoarthritis (Ziolkowska et al., The Journal of Immunology, 164:2832-2838 (2000)), and may deteriorate lesions of cartilage damage. For this reason, substances effective against rheumatoid arthritis cannot be predicted to have therapeutic efficacy on osteoarthritis, and their effects on cartilage regeneration are more difficult to predict.

In a process of cartilage destruction causing osteoarthritis, inflammation may be partially induced and lead to the release of inflammation-associated enzymes, thus accelerating the deterioration of cartilage. During the progress of osteoarthritis, synovial fluid volume increases with inflammation. This phenomenon involves the release of proteoglycan into synovial fluids due to the destruction of cartilage. Thus, a decrease in proteoglycan levels in synovial fluids is an important indicator of the improvement of osteoarthritis.

In addition, when osteoarthritis is induced, pain increases with inflammation of the joints. This increased pain is due to increased concentrations of prostaglandin E2, which is an important factor associated with the increased pain. Thus, it is also important to reduce prostaglandin E2 levels in synovial fluids so as to improve osteoarthritis.

With the progress of osteoarthritis, the number of synovial membrane lining cells in joint synovial fluids is increased, and the surface of synovial lining layers is not smooth and becomes thickened (Asari, A. et al., Arch. Histol. Cytol. 61 (2):125-35 (1998)).

Further, collagen is known to be associated with the incidence of arthritis. In particular, collagen is a material comprising articular cartilage, and its excess production promotes the hardness of cartilage and is related to the incidence of arthritis. Thus, higher than normal levels of collagen are used as an indicator of the incidence of osteoarthritis. Also, a decrease in this increased collagen levels is useful as an indicator of the improvement of osteoarthritis.

Therefore, it is very important in the treatment of osteoarthritis to decrease these tissue biochemical markers, and this decrease is indirect evidence for the improvement of damaged tissues.

Typically, excess NO is generated at inflammation sites and stimulates the necrosis of cellular tissues (Moncada, S. et al., Pharmacol. Rev. 43:109-142 (1991)). Thus, in the treatment of many inflammatory diseases, it is important to inhibit the activity or expression of iNOS (inducible nitric oxide synthase) responsible for NO generation. iNOS produces excess NO in a short time to defend the body against external stimuli, but, in diseases such as arthritis, excess production of NO brings about necrosis and secondary side effects such as pain. Thus, the inhibition of iNOS overexpression is important for the treatment of osteoarthritis.

Cyclooxygenase 2 (COX-2) is an enzyme that synthesizes a pain inducer, prostaglandin. Prostaglandin synthesis is stimulated by NO or other stimuli. Thus, the inhibition of the expression or activity of COX-2 is also an important indicator of the treatment of osteoarthritis.

In addition, NFκB is a transcription factor that is activated via a related signal pathway and initiates the gene expression of the aforementioned iNOS and COX-2. NFκB is sequestered in the cytoplasm in an inactive form bound to the inhibitor IκBα. Upon external stimulation such as inflammation, the IκBα inhibitor is phosphorylated and degraded. This then allows NFκB to translocate to the nucleus where it initiates transcription of the iNOS gene. Thus, the inhibition of phosphorylation or degradation of IκBα A present in the cytoplasm is an important indicator for evaluating the treatment of osteoarthritis.

Since NFκB is, as described above, sequestered in the cytoplasm in an inactive form and, upon external stimulation such as inflammation, enters the nucleus and initiate the gene transcription of iNOS and COX-2, the inhibition for NFκB to enter the nucleus and bind to a transcription regulatory element of a target gene is an important indicator for evaluating the improvement of osteoarthritis in patients with osteoarthritis.

Plant-derived naturally occurring flavonoids are phenolic compounds that are classified according to their structures into four groups: flavonols, flavanones, flavanols and flavans. Flavonoids have been known to be present in diverse forms in vegetables, fruits, teas, Chinese medicinal herbs, and the like, and to have a variety of effects in the body, including antiviral activity (e.g., Kaul, T. N. et al., J. Med. Virol. 15:71-79 (1985)), anticancer activity (e.g., Miller, A. B. et al., Rev. Oncol. 3:87-95 (1990)), anti-inflammatory activity (e.g., Ferrandiz, M. L. et al., J. Natl. Cancer Inst. 85:1038-1049 (1993)), and antioxidant activity (e.g., Cao, G. et al., Free Radi. Biol. Med. 22:749-760 (1997)).

One of the flavonoids, apigenin (4′,5,7-trihydroxyflavone), has the following chemical structure having a molecular weight of 270.24, and is a naturally occurring compound found in a large number of plants and fruits, including parsley (containing more than 0.05% apigenin) and thyme (containing more than 0.05% apigenin). Apigenin is known to have diverse biological activities including anti-inflammatory, vasordilatory, antioxidative, antiviral and anticancer actions.

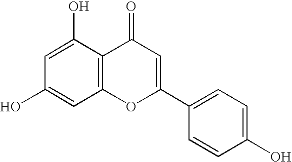

With respect to its anticancer activity, apigenin acts effectively even in a very low concentration, for example, lower than about 50 μM. Apigenin exhibits antiproliferative and cytotoxic effects by affecting apoptosis and necrosis mechanisms during cell proliferation and angiogenesis that are the major characteristics of a variety of cancer cells including prostate cancer, breast cancer, lung cancer, rectum cancer, blood cancer (leukemia), skin cancer, thyroid cancer and liver cancer, resulting in the inhibition of proliferation of cancer cells.

In addition, apigenin has cytotoxic and proliferation-inhibitory effects, such as accumulation of a tumor suppressor protein, p53, and apoptosis induction, in non-tumor cells such as murine embryo fibroblasts as well as cancer cells, and also has a slight proliferation-inhibitory effect on human normal prostate epithelial cells (Plaumann B. et al., Oncogene 13 (8), 1605-14 (1996) and Gupta, S. et al., Biochem. Biophys. Res. Commun. 287 (4):914-920).

However, according to a recent report, apigenin displays excellent efficacy in vitro where lung carcinoma and rectum carcinoma cell lines are treated with various concentrations of apigenin, but is rarely effective in vivo (Engelmann, C. et al., Phytomedicine 9 (6):489-495 (2002)). In particular, the effects of apigenin based on its proliferation-inhibitory activity include both proliferation inhibition and cytotoxicity and are very sensitive in low concentrations of apigenin. In contrast, various tests based on the antioxidative effect of flavonoids including apigenin revealed that flavonoids have protective or preventive effects against cytotoxicity (Wang, C. N. et al., J. Biol. Chem. 276 (7):5287-5295 (2001)).

The relation of apigenin to cartilage in arthritis is mentioned in U.S. Pat. No. 5,650,433 and its corresponding patents. The cited patents suggests that flavonoid compounds including apigenin and glycoside compounds are useful for treating arthritis because they delay the destruction of articular cartilage. However, in embodiments for this spirit and scope, where chondrocytes are treated with a flavonoid compound and a proteoglycan depleting agent, PMA (phorbol myristate acetate), the flavonoid compound is evaluated only for its inhibitory effect against proteoglycan depletion. That is, the cited patent describes the use of the flavonoid compound only as a chondroprotective agent, not for cartilage regeneration, and did not demonstrate that apigenin has a chondroregenerative effect. Also, in the cited patent, the degree of proteoglycan depletion was measured only by in vitro tests, and substantial in vivo effects of the flavonoid compound on chondroprotection and cartilage regeneration were not demonstrated in detail. Further, the cited patent describes arthropathy that includes osteoarthritis and rheumatoid arthritis, but describes, only in rheumatoid arthritis, the aforementioned chondroprotective effect of the flavonoid compound involving inhibition of proteoglycan depletion.

To date, there is no publication associated with the effects of apigenin on chondrocyte proliferation and cartilage regeneration in the joints or the application of accurate concentrations of apigenin to achieve these effects.

When cartilage damaged by the occurrence of osteoarthritis is to be regenerated, since an effect opposite to the antiproliferative and cytotoxic mechanisms of an anticancer action should be exerted, accurate concentrations of apigenin to achieve cytotoxic and antiproliferative effects on chondrocytes should be primarily determined in in vitro tests. Also, by in vivo tests where apigenin is applied to osteoarthritis patients, substantially effective concentrations of apigenin to exert an ability to proliferate chondrocytes with no toxicity should be determined.

To determine the relation between the potentiality of apigenin as a therapeutic agent for osteoarthritis and the conventionally-identified contrary cytotoxic and antiproliferative effects of apigenin, the present inventors first examined whether apigen has cytotoxic and antiproliferative effects on chondrocytes in vitro. As a result, in chondrocytes treated with apigenin in very low concentrations of lower than about 10 μm for one to two days, apigenin rarely exhibited cytotoxic and antiproliferative effects. Upon two-day and six-day cultures, the 50% inhibitory concentration (IC50) values for apigenin on chondrocytes were about 100 μM and about 30 μM, respectively. In addition, in a concentration of 1 to 10 μM, apigenin was found to have a proliferative effect of about 10% to 20%.

Based on these in vitro test results, the prevent inventors investigated the chondrocyte-proliferating effect of apigenin in an osteoarthritis-induced animal model.

Various effects of apigenin in osteoarthritis patients, including biochemical effects, histological effects, and chondroregenerative and synovial membrane-improving effects, were examined using New Zealand white rabbits as an osteoarthritis-induced animal model. New Zealand white rabbits underwent anterior cruciate ligament transection (ACLT) and were forced to sustain movement in a closed space to induce substantial osteoarthritis.

When various concentrations of apigenin were injected directly to affected sites of the osteoarthritis-induced rabbits, apigenin was found to exert a chondroregenerative effect in a dosage of about 0.1 to 100 μM, and preferably 1 to 80 μM, with no cytotoxicity. This concentration range of apigenin is not correlated with the results of in vitro cytotoxicity tests, and thus, it is hard to predict effective amounts of apigenin for chondroregeneration only by in vitro tests. In Example 1 of the present invention, the effects of apigenin on osteoarthritis were evaluated using an apigenin solution of about 80 μM (prepared by dissolving 50 μg of apigenin in 50 μl of DMSO and mixing the resulting solution with 450 μl of physiological saline)

In detail, a test group was injected with the aforementioned amounts of apigenin a total of four times (once per week), and a control group was injected with PBS. Synovial fluids were collected every two weeks, and changes in synovial fluid volume, as an osteoarthritis indicator, were measured.

In addition, biochemical tests were carried out. In the collected synovial fluids, changes in proteoglycan, total protein, prostaglandin and collagen levels, which are used as osteoarthritis markers, were measured. Also, the effect of apigenin on inflammation was evaluated by measuring changes in nitrous oxide (NO) levels as an inflammation marker in synovial fluids and measuring the effect of apigenin on LPS-induced NO production in a marcrophage cell line, RAW 264.7. Further, the effect of apigenin on prostaglandin production in the macrophage cell line was evaluated.

To determine whether apigenin regulates osteoarthritis marker proteins in gene levels, the effect of apigenin on the gene expression of iNOS, COX-2 and IκBα was evaluated. Also, the inhibitory effect of apigenin against the binding of NFκB to its nuclear target genes was evaluated.

In histological tests, degenerative changes, such as structural changes of cartilage, cell number changes in cartilage and surface staining distribution by cartilage staining with a dye, were evaluated according to a standard scoring system for the progress of osteoarthritis, Mankin's scoring (Mankin, H. J. et al., Orthopedic Clinics of North America, 2:19-30 (1971)). In the present invention, blind tests were performed by two inspectors according to the Mankin's criteria.

The above evaluations resulted in the finding that, in a dosage resulting in a concentration of 0.1 μM to 100 μM in an articular cartilage and a synovial fluid, apigenin has excellent biological activities in reducing the aforementioned biochemical indicators for the prevalence of osteoarthritis and, more importantly, has histological effects of stimulating cartilage regeneration as well as cartilage protection. Based on these results, the present invention provides a novel use of apigenin having a chondroregenerative effect as a chondroregenerative agent and a therapeutic agent for osteoarthritis comprising apigenin.

The terms used in the present specification have the following meanings.

The term “pharmaceutically acceptable carrier”, as used herein, refers to a carrier suitable for contact with human or animal tissue in a medically reasonable range while not causing unpredictable toxicity, irritation and allergies. Examples of the pharmaceutically acceptable carrier may include distilled water, isotonic saline, Ringer's solution and injectable water, each of which contains a small amount of an organic solvent allowing apigenin to be dissolved therein, such as DMSO.

The term “pharmaceutically acceptable excipient”, as used herein, refers to a nontoxic inert solid, semi-solid or liquid filler, a diluting agent, a capsulating material or a formulation adjuvant of a certain type. Examples of the pharmaceutically acceptable excipient may include lactose, glucose, sucrose; starches, such as corn starch and potato starch; cellulose and its derivatives, such as sodium carboxymethyl cellulose, ethyl cellulose and cellulose acetate; tragacanth; malt; gelatin; talc; cocoa butter; natural vegetable oils, such as arachis oil, cottonseed oil, safflower oil, sesame oil, olive oil, corn oil and soybean oil; glycols, such as polypropylene glycol; polyols, such as glycerin, sorbitol, mannitol and polyethylene glycol; esters, such as ethyl oleate and ethyl laurate; agar; buffers, such as magnesium hydroxide and aluminum hydroxide; alginic acid; distilled water; isotonic saline; Ringer's solution; ethyl alcohol; and phosphate buffers, and may further include other nontoxic substances suitable for use in pharmaceutical formulations.

In addition, examples of the excipient may further include humectants, emulsifiers and lubricants, as well as coloring agents, releasing agents, coating agents, sweetening agents, flavoring agents and aromatics.

The term “chondroregenerative effect”, as used herein, is intended to mean the effect of apigenin according to the present invention of stimulating the proliferation of chondrocytes as well as inhibiting the destruction of chondrocytes, with no cytotoxicity or cell proliferation inhibition.

With respect to the chondroregenerative effect, the term “an effective amount of apigenin for stimulating the proliferation of chondrocytes with no cytotoxicity” is intended to mean an effective amount of apigenin for stimulating proliferation of damaged chondrocytes without causing toxicity to a patient when the apigenin is administered to the patient requiring proliferation of chondrocytes. In accordance with the present invention, apigenin induces regeneration of articular cartilage in a concentration of about 0.1 to about 100 μM, preferably about 1 to about 80 μM, and more preferably about 1 to about 10 μM.

With respect to osteoarthritis treatment, the term “a therapeutically effective amount of a composition for regenerating cartilage” refers to a composition for regenerating cartilage, which contains apigen in an amount effective for improving osteoarthritis by regenerating damaged articular cartilage when the apigenin is administered to an osteoarthritis patient requiring cartilage regeneration.

In the present composition or formulation, a “therapeutically effective amount” may be determined by the judgment of supervising physicians within a medically acceptable range. A particular therapeutically-effective amount for a specific patient may vary depending on a variety of factors including the type of a composition or formulation; the patient's age, weight, health, gender and diet; administration duration and routes; therapy period; co-administered drugs; and other factors widely known in the medical field. For example, upon once administration per day at one week intervals, the present composition may be typically administered in a unit dosage of 10 mg to 1000 mg/day for oral administration, in a unit dosage of 0.1 mg to 10 mg/day in the case of injectable formulations, and in a unit dosage of 1 mg to 100 mg/day in the case of ointments, but the present invention is not limited to these examples. The above dosages illustrate average cases but may decrease or increase according to differences between individuals, and this modification is also included in the scope of the present invention.

The present composition or formulation may be administered to all animals liable to osteoarthritis, including humans.

The therapeutic agent for osteoarthritis according to the present invention is provided in a unit dosage form comprising the present composition for regenerating cartilage. In the case of injectable preparations, for example, sterile injectable aqueous or oily suspensions may be formulated using suitable dispersing agents or humectants and suspending agents according to a method known in the art. Sterile injectable preparations may be sterile injectable solutions, suspensions or emulsions in nontoxic non-orally acceptable carriers or solvents. Available vehicles and solvents include water, Ringer's solution and an isotonic sodium chloride solution. In addition, sterile hardened oil typically used as a solvent or suspension medium may be used. Injectable preparations may be injected intravenously, intracavernosally, intramuscularly, subcutaneously and intraductally.

For parenteral administration, a sterile aqueous solution form is most preferable. To be isotonic with blood, this solution may contain, for example, a material such as salts or sugars such as glucose and mannitol.

Solid dosage forms for oral administration may include capsules, tablets, pills, powders and granules. In these solid dosage forms, active compounds may be mixed with, for example, one or more inert diluents, such as sucrose, lactose or starch. Also, in addition to the inert diluents, additives, for example, lubricants and other adjuvants such as magnesium stearate and microcrystalline cellulose may be included. In the case of capsules, tablets and pills, the dosage forms may include buffering agents. Tablets and pills may further use enteric coating agents and other sustained-release coating materials. In soft and hard gelatin capsules, excipients such as lactose or milk sugar and high molecular weight glycols may be used as fillers.

Solid dosage forms including tablets, sugar coated tablets, capsules, pills and granules may be prepared using coating and shell-making systems.

Liquid dosage forms for oral administration may include pharmaceutically acceptable emulsions, solutions, suspensions, syrups and elixirs, which contain an inert diluent commonly used in the art, such as water. These compositions may include humectants, emulsifiers and suspending agents, sweetening agents, and flavoring agents and aromatics.

In addition, the formulation of the present invention may be prepared as ointments, pastes, creams, lotions, gels or patches in topical or transdermal administration dosage forms. Transdermal patches have an additional advantage of regulating the amount of a composition delivered to the body. These dosage forms may be prepared by dissolving or dispersing compounds in a suitable medium. An absorption stimulator may be used to increase the absorption of a compound through the skin. For transdermal administration, the present composition may be formulated into a pharmaceutically acceptable poultice containing sodium polyacrylic acid, glycerin and methylparaben, or a plaster containing propylene glycol, liquid paraffin and isopropyl myristate.

A better understanding of the present invention may be obtained through the following examples which are set forth to illustrate, but are not to be construed as the limit of the present invention.

### EXAMPLE 1

### The Antiproliferative and Cytotoxic Effects of Apigenin

Apigenin is known to have antiproliferative and cytotoxic effects on tumor cells as well as normal cells. Thus, to achieve the chondrocyte-proliferating effect of apigenin according to the present invention, apigenin was evaluated for its cytotoxicity on chondrocytes in vitro.

To determine the concentrations of apigenin effective for exerting its antiproliferating and cytotoxic effects on chondrocytes, IC₅₀ values were measured in chondrocytes isolated from the cartilage of New Zealand white rabbits. In brief, chondrocytes were isolated from normal cartilage of the rabbits by collagenase treatment, and treated with apigenin in various concentrations of 1, 10, 20, 30, 40, 50, 100 and 200 μM (containing lower than 0.05% DMSO). During culturing, on Days 1, 2, 4 and 6, IC₅₀ values were measured by a MTT assay based on cell viability.

As a result, IC₅₀ values for apigenin on a primary culture of the rabbit chondrocytes, indicating the antiproliferative effect of apigenin, are given in Table 1, below.

TABLE 1
IC₅₀ values for apigenin on chondrocytes

| Treated time | IC₅₀ values |
|---|---|
| 1 day | About 200 μM |
| 2 days | About 100 μM |
| 4 days | About 40 μM |
| 6 days | About 30 μM |

In particular, in chondrocytes cultured for one and two days in the presence of a very low concentration of apigenin, lower than about 10 μM, no significant proliferation inhibition or cytotoxicity was observed. Instead, in a concentration of 1 μM to 10 μM, apigenin displayed a cell proliferating effect of about 10% to 20% on chondrocytes. Thus, apigenin is expected to have a chondroregenerative effect in a concentration lower than 10 μm while stimulating proliferation of chondrocytes and minimizing its cytotoxicity in vitro.

These results demonstrate that, upon the treatment of osteoarthritis using apigenin, the determination of effective concentrations of apigenin is an important factor to achieve the anti-inflammatory and chondroregenerative effects of apigenin while avoiding the antiproliferative and cytotoxic effects of apigenin.

### EXAMPLE 2

### Osteoarthritis Induction in an Experimental Animal

To prepare an osteoarthritis animal model, New Zealand white rabbits underwent anterior cruciate ligament transection (ACLT) in joints of their two hind legs. After three days, the rabbits were forced to sustain movement for four weeks in a space of 5×5 m³ to induce substantial osteoarthritis. 50 μg of apigenin (A3145, Sigma) was administered a total of four times (once per week for four weeks) to affected right legs of the osteoarthritis-induced rabbits. An injectable preparation of apigenin was prepared by dissolving 50 μg of apigenin in a mixture of 50 μl of DMSO (dimethylsulfoxide) and 450 μl of physiological saline. Affected left legs were used as a control for the affected right legs, and injected with 450 μl of a physiological buffer, PBS, containing 50 μl of DMSO so as to compare to the effects of apigenin. One week after apigenin was administered in Week 2 and Week 4 after osteoarthritis induction, joint synovial fluids and cartilage were collected and used in tests. FIG. 13 shows the process of osteoarthritis induction and synovial fluid collection. The following Examples 3 to 10 were all performed using joints of the osteoarthritis-induced New Zealand white rabbits prepared as in this Example 2.

FIG. 1 photographically shows the affected joints of test and control groups. As shown in FIG. 1, in rabbits administered with an effective amount of apigenin according to the present invention, the surface of joint cartilage was visually smooth. In contrast, in the PBS control group, the surface of joint cartilage was very rough due to the damage of the joint cartilage. In addition, the cartilage in the left joint of the test group administered with apigenin was found to be improved to some extent due to decreased weight owing to the improvement of the cartilage in a right joint.

### EXAMPLE 3

### Measurement of Total Joint Synovial Fluid Volume

Typically, synovial fluid volume increases with inflammation as osteoarthritis progresses. Before osteoarthritis (OA) induction (normal state) and one week after apigenin was administered in Week 2 and Week 4 after osteoarthritis induction, synovial fluids were collected and centrifuged to remove blood cells and other cells. The supernatants were used in this test.

Since synovial fluid volume has been reported to change according to the progress of osteoarthritis, total synovial fluid volume was measured to investigate changes in synovial fluids according to osteoarthritis induction and drug treatment. Synovial fluid volume was determined by measuring Ca²⁺ concentrations in synovial fluids using an Arsenazo III complexion method (Michaylova V. et al., Anal. Chim. Acta, 53:194 (1971)). Total synovial fluid volume was calculated using a Donnan equilibrium equation. 0.01 ml of a synovial fluid was mixed with 1 ml of an Arsenazo III reagent (588-3, Sigma) and allowed to stand at room temperature for 5 min. Absorbance was measured at 600 nm using a spectrophotometer (DU650, Beckman).

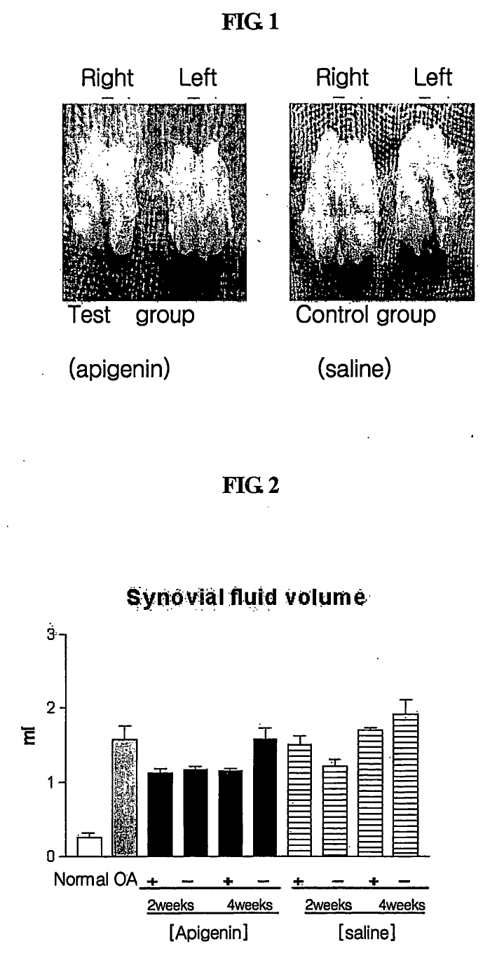

The results are given in Table 2, below, and FIG. 2.

Four weeks after drug injection, a normal total synovial fluid volume (before OA induction) of about 0.25 ml was increased to about 1.7 ml in a PBS control group. In contrast, an apigenin treatment group displayed a lower increase in total synovial fluid volume to about 1.16 ml four weeks after drug injection, indicating that apigenin has a strong inhibitory effect against an increase in synovial fluid volume. In addition, in the apigenin treatment group, an affected left joint (not treated with apigenin) displayed a lower synovial fluid volume of about 1.58 ml four weeks after the apigenin injection than the PBS control group having a synovial fluid volume of about 1.91 ml. This improvement in the affected left joint was due to decreased weight owing to the right joint having been improved by apigenin injection.

TABLE 2
Total synovial fluid volume (ml)

| | | Four weeks after | Apigenin | Two weeks after | Four weeks after |
|---|---|---|---|---|---|
| | Normal | OA induction | treatment | drug injection | drug injection |
| Apigenin | 0.25 ± 0.12 | 1.59 ± 0.32 | +(Right leg) | 1.13 ± 0.08 | 1.16 ± 0.03 |
| treatment group | | | −(Left leg) | 1.17 ± 0.08 | 1.58 ± 0.24 |
| PBS | 0.26 ± 0.09 | 1.75 ± 0.11 | +(Right leg) | 1.5 ± 0.22 | 1.7 ± 0.05 |
| control group | | | −(Left leg) | 1.2 ± 0.16 | 1.91 ± 0.34 |

### EXAMPLE 4

### Measurement of Proteoglycan Levels in Joint Synovial Fluids

In the joint synovial fluids prepared in Example 3, proteoglycan levels were measured using a 1,9-dimethylmethylene blue assay (Houselmann H. J. et al., Am. J. Physiol. 271:C742-752, (1996)). A synovial fluid sample of 50 μl was mixed with 250 μl of 1,9-dimethylmethylene blue (34,108-8, Aldrich), and absorbance was measured at 530 nm using a spectrophotometer (Power Wave X340, Bio-Tek).

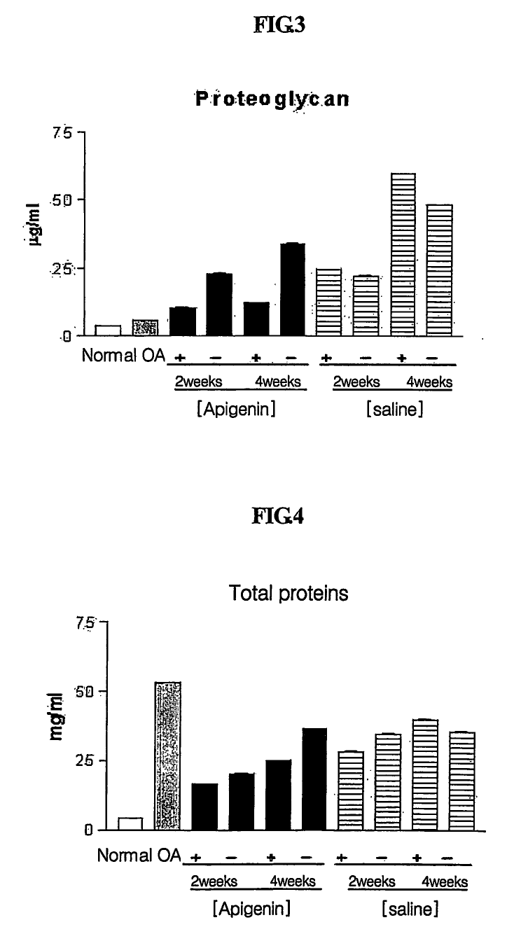

The results are given in Table 3, below, and FIG. 3. Four weeks after drug injection, a normal proteoglycan level (before OA induction) of about 3.79 μg/ml was increased to about 59.65 μg/ml in a PBS control group. In contrast, compared to the control group, an apigenin treatment group displayed a sharp decrease in proteoglycan levels to about 12.22 μg/ml four weeks after drug injection, indicating that apigenin has a strong effect on decreasing proteoglycan levels in OA-induced joints. In addition, in the apigenin treatment group, an affected left joint (not treated with apigenin) displayed lower proteoglycan levels than the PBS control group.

TABLE 3
Proteoglycan levels (μg/ml) in joint synovial fluids

| | | Four weeks after | Apigenin | Two weeks after | Four weeks after |
|---|---|---|---|---|---|
| | Normal | OA induction | treatment | drug injection | drug injection |
| Apigenin | 3.79 ± 0.07 | 5.84 ± 0.44 | +(Right leg) | 10.32 ± 0.66 | 12.22 ± 0.4 |
| treatment group | | | −(Left leg) | 22.90 ± 0.86 | 34.09 ± 0.24 |
| PBS | 3.38 ± 0.29 | 3.24 ± 0.42 | +(Right leg) | 24.42 ± 0.40 | 59.65 ± 0.52 |
| control group | | | −(Left leg) | 22.25 ± 0.22 | 48.47 ± 0.49 |

### EXAMPLE 5

Measurement of Total Protein Levels in Joint Synovial Fluids

As cartilage is destroyed with the progress of osteoarthritis, proteins contained therein are released into synovial fluids. With this respect, total protein levels were measured in synovial fluids. Using the joint synovial fluids prepared in Example 3, total protein levels in synovial fluids were measured using a Bradford method. 50 μl of a synovial fluid was mixed with 200 μl of a protein analysis reagent (500-0006, Bio-Rad) and allowed to stand at room temperature for 5 min, and absorbance was then measured at 595 nm.

The results are given in Table 4, below, and FIG. 4.

Four weeks after drug injection, a normal total protein level (before OA induction) of about 4.27 mg/ml increased to about 39.74 mg/ml in a PBS control group. In contrast, compared to the control group, an apigenin treatment group displayed a significant decrease in total protein levels to about 24.91 mg/ml four weeks after drug injection.

TABLE 4
Total protein levels (mg/ml) in joint synovial fluids

| | | Four weeks after | Apigenin | Two weeks after | Four weeks after |
|---|---|---|---|---|---|
| | Normal | OA induction | treatment | drug injection | drug injection |
| Apigenin | 4.27 ± 0.24 | 53.24 ± 0.24 | +(Right leg) | 16.64 ± 0.3 | 24.91 ± 0.35 |
| treatment group | | | −(Left leg) | 20.36 ± 0.69 | 36.37 ± 0. |
| PBS | 4.29 ± 0.68 | 53.64 ± 0.47 | +(Right leg) | 28.02 ± 0.83 | 39.74 ± 0.52 |
| control group | | | −(Left leg) | 34.69 ± 0.36 | 35.28 ± 0.53 |

### EXAMPLE 6

### Measurement of Prostaglandin E2 (PGE2) Levels in Joint Synovial Fluids

Using the joint synovial fluids prepared in Example 3, prostaglandin E2 (PGE2) levels in synovial fluids were measured using an enzyme immunoassay (EIA) kit (DE0100, R&D Systems). 100 μl of a 1:10 dilution of a synovial fluid sample was mixed with 50 μl of a conjugate and 50 μl of an antibody in a plate provided in the kit, and was incubated with agitation at room temperature for two hours. Then, the plate was washed with a washing buffer, and 200 μl of a pNPP solution was added thereto, followed by incubation at room temperature for one hour. Thereafter, absorbance was measured at 405 nm.

The results are given in Table 5, below, and FIG. 5. Four weeks after drug injection, a normal PGE2 level (before OA induction) of about 88.05 pg/ml increased to about 862.72 pg/ml in a PBS control group. In contrast, in an apigenin treatment group, the normal PGE2 level slightly increased to about 196.71 pg/ml, thus being greatly reduced in comparison with the control group. These results indicate that apigenin has a strong inhibitory effect against an increase in PGE levels. In addition, in the apigenin treatment group, an affected left joint (not treated with apigenin) displayed a significantly decreased PGE2 level of about 753.2 pg/ml in comparison with the PBS control group having a PGE2 level of about 509.3 pg/ml.

TABLE 5
PGE2 levels (pg/ml) in joint synovial fluids

| | | Four weeks after | Apigenin | Two weeks after | Four weeks after |
|---|---|---|---|---|---|
| | Normal | OA induction | treatment | drug injection | drug injection |
| Apigenin | 88.05 ± 2.08 | 503.98 ± 1.41 | +(Right leg) | 183.26 ± 0.2 | 196.71 ± 0.52 |
| treatment group | | | −(Left leg) | 543.3 ± 1.13 | 509.2 ± 9.2 |
| PBS | 90.6 ± 0.71 | 503.51 ± 0.72 | +(Right leg) | 685.7 ± 0.54 | 862.7 ± 0.36 |
| control group | | | −(Left leg) | 571.7 ± 0.57 | 753.2 ± 0.31 |

### EXAMPLE 7

### Measurement of Collagen Levels in Joint Synovial Fluids

Total collagen levels in synovial fluids were measured by Sirius Red staining (Heide T. R. et al., Histochem. Cell Biol., 112: 271-276 (1999)). 100 μl of a synovial fluid sample was placed into each well of a 96-well plate and incubated in a dry oven at 34° C. for 24 hrs to coat the bottom of wells. 100 μl of a dye solution, prepared by dissolving Sirius Red (1 mg/ml, 36,554-8, Aldrich) in picric acid, was added to each well, and the plate was agitated for 30 min. The remaining dye was removed using 0.01N HCl (H7020, Sigma), and collagen was dissolved using 100 μl of 0.1N NaOH (S8045, Sigma). Then, total collagen levels were determined by measuring absorbance at 550 nm. A standard curve was obtained by measuring absorbance at 550 nm for collagen type I (C1188, Sigma) of various concentrations of 0, 100, 200, 300, 400 and 500 μg. Using the standard curve, collagen levels in synovial fluids were determined.

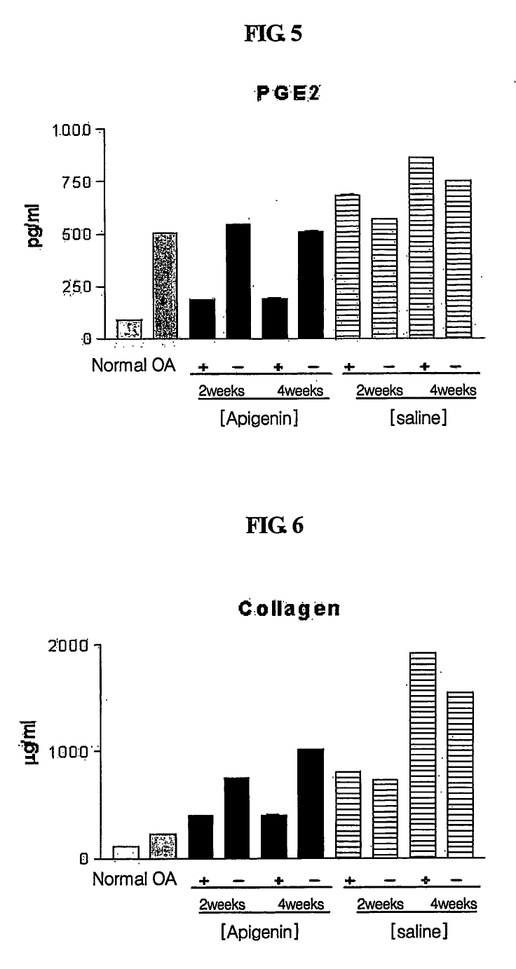

The result are given in Table 6, below, and FIG. 6. Four weeks after drug injection, a normal collagen level of about 118.64 μg/ml increased to about 1912.54 μg/ml in a PBS control group. In contrast, an apigenin treatment group showed a greatly reduced collagen level of 406.56 μg/ml, indicating that apigenin significantly inhibits collagen levels. In addition, in the apigenin treatment group, an affected left joint (not treated with apigenin) displayed a significantly decreased collagen level in a synovial fluid of about 1019.2 μg/ml in comparison with the PBS control group having a PGE2 level of about 1551 μg/ml.

TABLE 6
Collagen levels (μg/ml) in joint synovial fluids

| | | Four weeks after | Apigenin | Two weeks after | Four weeks after |
|---|---|---|---|---|---|
| | Normal | OA induction | treatment | drug injection | drug injection |
| Apigenin | 118.64 ± 0.64 | 233.18 ± 1.03 | +(Right leg) | 397.4 ± 0.69 | 406.6 ± 0.63 |
| treatment group | | | −(Left leg) | 749.4 ± 0.46 | 1019.2 ± 0.79 |
| PBS | 119.7 ± 0.44 | 233.38 ± 0.93 | +(Right leg) | 805.9 ± 0.19 | 1912.5 ± 0.7 |
| control group | | | −(Left leg) | 734.2 ± 0.71 | 1551 ± 0.64 |

### EXAMPLE 8

### Mankin's Scoring of Joints

30-mm synovial tissues were sectioned from the medial parapatella synovium of knee joints of experimental animals, fixed with 10% formic acid (F0507, Sigma) for over 24 hrs, and embedded in paraffin to make 4-mm paraffin blocks. The 4-mm sections were subjected to H&E (hematoxylin & eosin) staining and observed under an optical microscope to determine Mankin scores. Among several methods used for evaluating the progressed degree of osteoarthritis, the most commonly used method is Mankin's scoring. This method is based on expressing numerically degenerative changes as grades in each item, including structural changes of cartilage, an increase or decrease in cell number in cartilage, surface staining distribution by cartilage staining with safranin-O and continuance of tidemark. Mankin's scoring was performed by blind tests carried out by two inspectors, and Mankin scores were determined according to the degree of degenerative progress and ranged from 0 (normal in all items) to a maximum score of 14, thus calculating a degenerative change index.

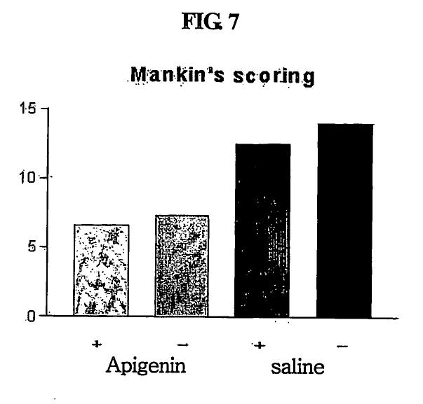

The results are given in FIG. 7. After osteoarthritis induction, mean scores in control and apigenin treatment groups were 12.5 and 6.6, respectively. That is, the apigenin treatment group displayed a two-fold improvement in a degenerative change index compared to the control group.

### EXAMPLE 9

### H&E Staining and Safranin-O Staining of Joints

Distal femurs were collected from experimental animals and fixed with 4% formalin (F8775, Sigma) for over 24 hrs. An area of the distal femur to be analyzed was sectioned into a 5-mm thickness, decalcified with 5% nitric acid (25, 811-3, Sigma) for over 24 hrs and embedded into paraffin to provide a paraffin block. The paraffin block was sectioned, and the resulting 4-μm sections were subjected to H&E staining and staining with a cartilage-specific dye, safranin-O (S2255, Sigma), and observed under an optical microscope to evaluate the condition of cartilage.

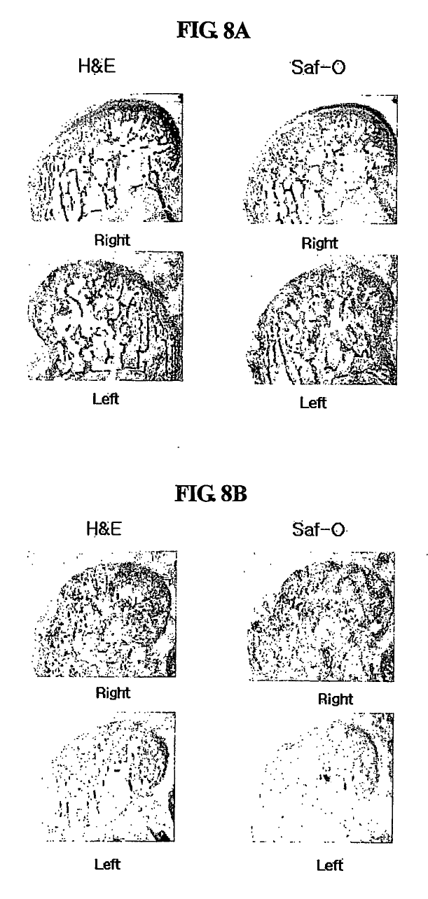

The results are given in FIGS. 8A and 8B, which show the results of H&E staining and safranin-O staining in an apigenin treatment group and a control group treated with physiological saline, respectively. Upon safranin-O staining, overall osteoarthritis features were observed, but, in the apigenin treatment group, cartilage destruction was low enough to be visually identified and showed a relatively high staining in proteoglycan, compared to the control group. When the apigenin treatment group was subjected to histological analysis for visually evaluating the cartilage-specific ECM synthesis such as proteoglycan and cell morphology, damaged areas of cartilage were found to be restored.

### EXAMPLE 10

### Evaluation of Distribution and Number of Synovial Cells

Tissues were prepared according to the same method as in Example 9 and evaluated for cell number and the condition of their surfaces. Cell number was measured at a site 120 μm in depth.

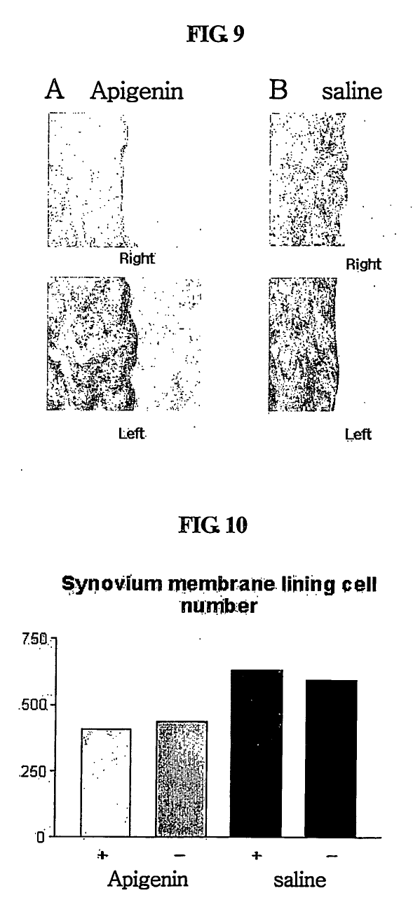

The results are given in FIGS. 9 and 10. As shown in FIG. 9, an apigenin treatment group had a relatively smooth surface in a section of a right leg in comparison with that of a control group treated with physiological saline. In addition, as shown in FIG. 10, after osteoarthritis induction, the number of synovial cells was 632.3 in the control group, while being 406.6 in the apigenin treatment group. That is, compared to the control group, the apigenin treatment group had a significantly decreased synovial cell number of about 34.5%.

### EXAMPLE 11

### Measurement of Nitric Oxide (NO) Levels in Synovial Fluids

To determine NO levels in synovial fluids, nitric acid was reduced to the most stable form, nitrite, and nitrite was measured using a Griess reaction. A synovial fluid and a Griess reagent were mixed at a ratio of 1:1 (100 μl: 100 μl) and allowed to stand at room temperature for 10 min, and absorbance was measured at 540 nm using a spectrophotometer (Power Wave X340, Bio-Tek). The Griess reagent was prepared in a final volume of 10 ml using 0.1 g of sulfanilamide (S9251, Sigma), 0.5 ml of phosphoric acid (P6560, Sigma) and 0.01 g of N-naphthyl-diamine-H-chloride (102397, ICN). A quantitative curve for nitrite was obtained using sodium nitrite (S2252, Sigma).

The results are given in Table 7, below. After osteoarthritis induction, a normal NO level (before OA induction) of about 4.48 μM increased to about 12.1 μM in a PBS control group, and slightly increased to about 6.01 μM in an apigenin treatment group. These results indicate that apigenin has a good anti-inflammatory effect.

TABLE 7
NO levels (μM) in synovial fluids

| | | Four weeks after | Apigenin | Two weeks after | Four weeks after |
|---|---|---|---|---|---|
| | Normal | OA induction | treatment | drug injection | drug injection |
| Apigenin | 4.48 ± 3.18 | 12.11 ± 0.88 | +(Right leg) | 7.53 ± 4.66 | 6.01 ± 0.88 |
| treatment group | | | −(Left leg) | 0.65 ± 0.06 | 5.5 ± 4.59 |
| PBS | 4.48 ± 3.18 | 12.11 ± 0.88 | +(Right leg) | 10.58 ± 2.33 | 12.1 ± 6.66 |
| control group | | | −(Left leg) | 0.65 ± 0.05 | 6 ± 0.88 |

### EXAMPLE 12

### The Inhibitory Effect of Apigenin on NO Production of Macrophages

A macrophage cell line derived from mice, RAW 264.7 (KCLB 40071), was purchased from the Korean Cell Line Bank. RAW 264.7 cells were seeded in a density of 3×10⁶ cells onto 60-mm culture dishes containing DMEM (Dulbecco's Modified Eagle Medium, 12800-017, Gibco) supplemented with 10% FBS (fetal bovine serum, 26140-079, Gibco), 100 U/ml of penicillin and 100 μg/ml streptomycin (15140-122, Gibco), and cultured at 37° C. under 5% CO₂ and humidity for 24 hrs. To induce NO production, RAW 264.7 cells were treated with 500 ng/ml of an E. coli cell wall component, lipopolysaccharide (LPS, L2654, Sigma). A control group was treated with only DMSO, and a test group was treated with apigenin in concentrations of 10, 20, 40 and 80 μM for 16 hrs. Herein, LPS was dissolved in distilled water, and apigenin was dissolved in DMSO. Then, the culture medium and a Griess reagent were mixed at a ratio of 1:1 (100 μl: 100 μl) and allowed to stand at room temperature for 10 min. Thereafter, absorbance was measured at 540 nm using a spectrophotometer (Power Wave X340, Bio-Tek).

The results are given in Table 8, below. A non-treatment group produced 2 μM nitric oxide, and a group treated with only LPS generated 40 μM nitric oxide. In contrast, in groups treated with LPS and apigenin, NO generation decreased to a maximum of 9 μM according to the concentrations of apigenin, indicating that apigenin has an excellent anti-inflammatory effect in vitro.

TABLE 8
Nitric oxide levels in RAW 264.7 cells

| Treatment | Nitrite (μM) |
|---|---|
| Non-treated | 2 |
| LPS (500 ng/ml) | 40 |
| LPS + apigenin (10 μM) | 32.2 |
| LPS + apigenin (20 μM) | 22.2 |
| LPS + apigenin (40 μM) | 10.1 |
| LPS + apigenin (80 μM) | 9 |

### EXAMPLE 13

### The Inhibitory Effect of Apigenin on Prostaglandin E2 (PGE2) Production of Macrophages

RAW 264.7 cells were treated under the same conditions as in Example 12. PGE2 levels were measured using a PGE2 immunoassay kit (DE0100, R&D Systems). 100 μl of a culture medium was allowed to react with 50 μl of a conjugate and 50 μl of an antibody in the kit for two hours, and was then allowed to react with 200 μl of a developing reagent, pNPP, for one hour. Thereafter, absorbance was measured at 405 nm using a spectrophotometer (Power Wave X340, Bio-Tek).

The results are given in Table 9, below. A non-treatment group produced 71 pg/ml of PGE2, and a group treated with only LPS generated 4156 pg/ml of PGE2. In contrast, in groups treated with LPS and apigenine, PGE2 production decreased to a maximum of 300 pg/ml according to the concentrations of apigenine.

TABLE 9
PGE2 levels in RAW 264.7 cells

| Treatment | PGE2 (pg/^{ml}) |
|---|---|
| Non-treated | 71 |
| LPS (500 ng/ml) | 4156 |
| LPS + apigenin (10 μM) | 3935 |
| LPS + apigenin (20 μM) | 3918 |
| LPS + apigenin (40 μM) | 1057 |
| LPS + apigenin (80 μM) | 300 |

### EXAMPLE 14

### Evaluation of the Effects of Apigenin on the Expression of iNOS, COX-2, IκBα in Macrophages

RAW 264.7 cells were treated under the same conditions as in Example 12. To investigate the effects of apigenin on the expression of iNOS, COX-2 and IκBα in macrophages, Western blotting was carried out. Herein, for IκBα expression, the cells were treated with LSP for only two hours. After media were removed, cells were collected and lysed with an extraction buffer (0.32 M sucrose (S0389, Sigma), 0.2 M Hepes (H3375, Sigma), 1 mM EDTA (808288, BM), 1 mM PMSF (P7626, Sigma), 10 μg/ml aprotinin (A1153, Sigma), 10 μg/ml leupeptin (L0649, Sigma), 10 μg/ml SBTI (T9128, Sigma)). After protein concentrations in cell lysates were measured, 40-mg protein samples were separated on an 8-16% SDS (sodium dodecyl sulfate) polyacrylamide gel (EC60452, NOVEX). The separated proteins were transferred onto a PVDF (polyvinylidene difluoride, IPVH00010, Millipore). The blot was blocked in 5% NFDM (non fat dry milk) and reacted with a primary antibody and then a secondary antibody. The blot was then developed using an ECL (enhanced chemiluminescence) kit (RPN2106, Amersham) and exposed to an X-ray film (AGFA). The following primary antibodies were used: 0.13 μg/ml of anti-iNOS (N32020, Transduction), 2 μg/ml of anti-COX-2 (sc-1745, Santacruz), 1 μg/ml of anti-β-actin (A5441, Sigma), and 0.4 μg/ml of anti-IκBα (sc-371, Santacruz). 80 ng/ml of anti-mouse IgG-HRP (sc-2005, Santacruz) was used as a secondary antibody for anti-iNOS and anti-β-actin. 80 ng/ml of anti-rabbit IgG-HRP (sc-2004, Santacruz) was used as a secondary antibody to anti-COX-2 and anti-IκBα.

The results are given in FIG. 11. Under normal conditions with no external stimulation, no iNOS expression was observed. An increased iNOS expression level upon LPS treatment was expressed as 100%. Upon apigenin treatment, the increased iNOS expression level of 100% decreased to 0%. Under normal conditions with no external stimulation, no COX-2 expression was observed. An increased COX-2 expression level upon LPS treatment was expressed as 100%. Upon apigenin treatment, the increased COX-2 expression level of 100% decreased to 0%.

Under normal conditions with no external stimulation, an IκBα expression level was expressed as 100%. LPS treatment resulted in a decrease to 18.56%. Upon treatment with only 10 μM of apigenin, the decreased IκBα expression level recovered to 75.82%.

### EXAMPLE 15

### Evaluation of the Effect of Apigenin on the Binding of NFκB to a Specific Gene in Macrophages

RAW 264.7 cells were treated under the same conditions as in Example 12 except treatment lasted for 2 hrs instead of 16 hrs. To investigate the effect of apigenin on the binding between NFκB and a specific gene in macrophages, EMSA (electrophoretic mobility shift assay) was carried out. After media were removed, cells were collected, lysed with Buffer A (10 mM Hepes, pH 7.9 (H3375, Sigma), 10 mM KCl (P9541, Sigma), 1.5 mM MgCl₂ (M2393, Sigma), 0.5 mM dithiothreitol (D0632, Sigma), 0.2 mM PMSF (P7626, Sigma), 0.5% Nonidet P-40 (N3268, Sigma)), and centrifuged at 5000 rpm for 15 min. The pellet was suspended to destroy the nuclear membrane in Buffer B (20 mM Hepes, pH 7.9 (H3375, Sigma), 300 mM KCl (P9541, Sigma), 1.5 mM MgCl₂ (M2393, Sigma), 10% glycerol (G7757, Sigma), 0.5 mM dithiothreitol (D0632, Sigma), 0.2 mM EDTA (808288, BM), 0.2 mM PMSF (P7626, Sigma)), and centrifuged at 13000 rpm for 30 min to obtain nuclear proteins. A synthesized nucleic acid oligomer (5′-AGT TGA GGG GAC TTT CCC AGG C-3′, GenoTech) that was able to bind to NFκB was labeled with [γ-³²P]ATP (PB10218, Amersham). A 10-mg protein sample was allowed to react with 0.5 ng of the labeled nucleic acid oligomer. Then, to evaluate the binding between the probe and NFκB, reaction solutions were subjected to 6% polyacrylamide gel electrophoresis, and the gel was dried and exposed to an X-ray film.

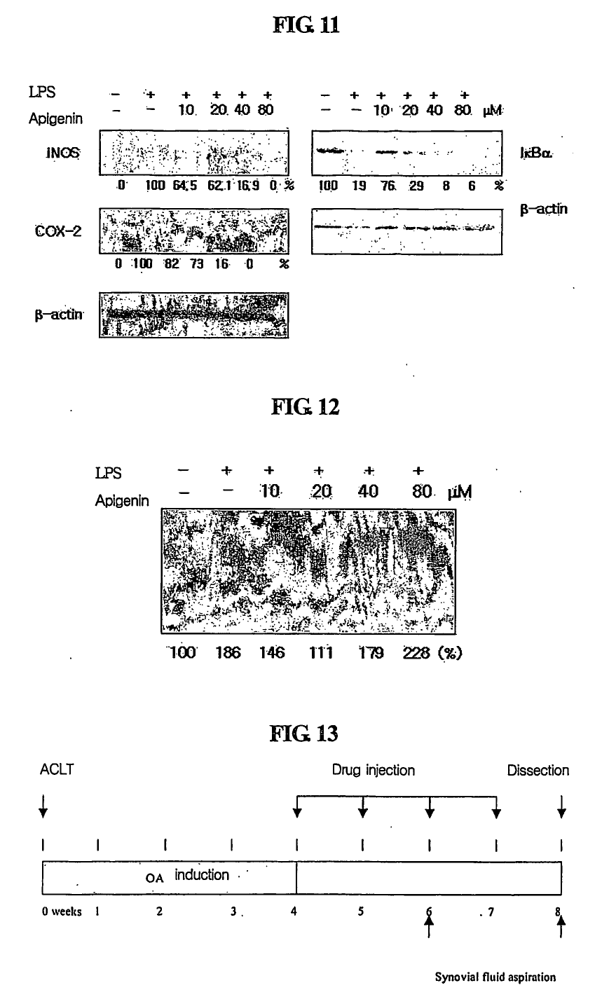

The results are given in FIG. 12. Under normal conditions with no external stimulation, an NFκB expression level was expressed as 100. LPS treatment resulted in an increase to 186. In contrast, upon treatment with both LPS and apigenin, the normal NFκB expression level decreased to a maximum of 111 (upon treatment of 20 μm apigenin).

### INDUSTRIAL APPLICABILITY

As described hereinbefore, the present invention provides a novel use of apigenin as a chondroregenerative agent, which has the effects of reducing elevated levels of cartilage destruction markers including total synovial fluid volume and proteoglycan, total proteins and prostaglandin in a synovial fluid, improving the condition of synovial cells, and regenerating cartilage. Also, the present invention provides a therapeutic agent for osteoarthritis comprising a single compound, apigenin, as an agent regenerating articular cartilage, and a method of treating osteoarthritis using such a therapeutic agent.

1. A composition for regenerating cartilage, comprising apigenin in an effective amount resulting in a concentration of 1 μM to 80 μM in an articular cartilage and a synovial fluid and a pharmaceutically acceptable carrier.

2. The composition for regenerating cartilage according to claim 1, wherein the composition has effects of reducing joint synovial fluid volume and levels of proteoglycan, total proteins and prostaglandin in a synovial fluid, and improving the condition of synovial cells.

3. A therapeutic agent for osteoarthritis for regenerating cartilage, comprising the composition for regenerating cartilage according to claim 1 and a pharmaceutically acceptable excipient.

4. The therapeutic agent for osteoarthritis according to claim 3, wherein the therapeutic agent is in,a form of orally administrable preparations of solutions, capsules, granules, tablets or pills; topically applicable preparations of ointments or transdermally administrable preparations; or injectable preparations.

5. The therapeutic agent for osteoarthritis according to claim 4, wherein therapeutic agent is in a form of the orally administrable preparations that contain apigenin in a daily effective amount of 10 mg to 1000 mg.

6. The therapeutic agent for osteoarthritis according to claim 4, wherein therapeutic agent is in a form of the ointments that contain apigenin in a daily effective amount of 1 mg to 100 mg.

7. The therapeutic agent for osteoarthritis according to claim 4, wherein therapeutic agent is in a form of the injectable preparations that contain apigenin in a daily effective amount of 0.1 mg to 10 mg.

8. A method of treating osteoarthritis by regenerating articular cartilage, comprising administering a therapeutic agent for osteoarthritis, comprising the composition for regenerating cartilage according to claim 1 and a pharmaceutically acceptable excipient, to an osteoarthritis patient.
